(19) United States
(12) Patent Application Publication
(10) Pub. No.: US 2007/0059701 A1
(43) Pub. Date: Mar. 15, 2007

(54) Fluorescence assays for nucleic acid polymerase activity

(75) Inventor: Adam Shapiro, WALTHAM, MA (US)

Correspondence Address: ASTRAZENECA R&D BOSTON, 35 GATEHOUSE DRIVE, WALTHAM, MA, 02451-1215, US

(73) Assignee: ASTRAZENECA AB, SODERTALJE (SE)

(21) Appl. No.: 10/561,307

(22) PCT Filed: Jun. 24, 2004
(86) PCT No.: PCT/GB04/02684

§ 371 Date: Dec. 19, 2005

Related U.S. Application Data
(60) Provisional application No. 60/484,031, filed on Jun. 30, 2003.

(51) Int. Cl. C12Q 1/68; C12P 19/34

(52) U.S. Cl. 435/6; 435/91.2

(57) ABSTRACT

The present invention provides methods of detecting nucleic acid polymerase activity and methods of detecting compounds that modulate nucleic acid polymerase activity.

### CROSS-REFERENCE TO RELATED APPLICATIONS

This application is related to U.S. Provisional Application No. 60/484,031, filed Jun. 30, 2003, which is incorporated herein by reference in its entirety.

### FIELD OF THE INVENTION

The present invention relates to methods of detecting nucleic acid polymerase activity and methods of detecting compounds that modulate nucleic acid polymerase activity.

### BACKGROUND

DNA polymerases are enzymes that covalently add nucleotides to a DNA or RNA primer in a template-directed fashion, i.e., the nucleotides added are complementary to the template sequence. The traditional method of assaying polymerase activity is to measure the incorporation of radioactively labeled nucleotides into acid-precipitable DNA. Other methods that have been described are (a) measuring the increase in fluorescence of a dye that has greater fluorescence when bound to double-stranded DNA than to single-stranded DNA or when unbound (Seville et al., 1996, BioTechniques, 21:664-672), and (b) measuring the increased intrinsic fluorescence of single-stranded DNA binding protein (SSB) when it is displaced from the DNA by synthesis of the complementary strand (Griep, 1995, Anal. Biochem., 232:180-189). Polymerase activity has also been assayed using time-resolved resonance energy-transfer, where the incorporated nucleotides are labeled (WO 01/38587).

Measurement of DNA denaturation by separation of FRET probes has been reported (Hiyoshi & Hosoi, 1994, Anal. Biochem., 221:306-311). FRET has also been applied to monitoring helicase-catalyzed unwinding of duplex DNA (Bjornson et al., 1994, Biochemistry 33, 14306-14316), and to the study of DNA cleavage (Lee et al., 1997, Methods Enzymol., 278:343-363).

### SUMMARY

The present invention is based, in part, on the finding that the stability of a double stranded nucleic acid product in the presence of a denaturant can be used as a means for investigating the activity of a nucleic acid polymerase. The method of the present invention can be used to investigate the activity of a nucleic acid polymerase and to identify compounds that modulate nucleic acid polymerase activity.

Accordingly, the method of detecting nucleic acid polymerase activity includes providing a primer-template hybrid duplex comprising a nucleic acid template and a nucleic acid primer, wherein the template, the primer, or both the template and the primer comprise a label. The duplex is then contacted with a nucleic acid polymerase under conditions which allow the nucleic acid polymerase to elongate the primer. The duplex is then subjected to denaturing conditions and a signal from the label is detected. A change in the signal compared to a control is indicative of nucleic acid polymerase activity.

As above, the template, the primer, or both the template and the primer can include a label. The label can be any detectable group such as ³²P, or any other detectable marker, such as other radioisotopes, a fluorescent compound, an enzyme, or an enzyme co-factor. In one example, the template and the primer are labeled such that when they are in close proximity a signal is generated. For example, the nucleic acid template includes a first label that can be a fluorescence donor and the template includes a second label which can be a FRET acceptor or fluorescence quencher. In another embodiment, the first label can be a FRET acceptor or fluorescence quencher and the second label is a fluorescence donor. An example of a fluorescence donor is 5- or 6-carboxyfluorescein (FAM) and an example of a FRET acceptor is 5- or 6-carboxytetramethylrhodamine (TAMRA).

The nucleic acid template or the primer can be in solution or can be immobilized to a surface. In one embodiment, the nucleic acid template is immobilized close to a scintillant molecule and the primer is labeled with a radioisotope. In another example, the primer can be immobilized close to a scintillant molecule and the nucleic acid template is labeled with a radioisotope.

The method of the present invention can be used to determine the activity of any nucleic acid polymerase. In one example, the nucleic acid polymerase is a DNA polymerase from a prokaryote or a eukaryote. In one embodiment, the DNA polymerase is bacterial DnaE. Examples of DNA polymerases include E. coli DnaE or H. influenzae DnaE.

Appropriate denaturing conditions can be achieved by providing conditions that result in the primer-template duplex disassociating should the test nucleic acid polymerase not have sufficient activity. The denaturant can be a chaotropic agent such as urea or the denaturant can be a change in the reaction environment such as the application of heat.

The label can be attached to the primer and/or template at any appropriate location. In one embodiment, the first label is borne at the 5′ end of the primer and the second label is borne at the 3′ end of the template.

The primer can be of any appropriate length such that it hybridizes to the template. In one example, the primer can be at least 6 nucleotides in length and the template can be at least 10 nucleotides in length.

The invention also includes a method of screening for compounds such as peptides, peptidomimetics, small molecules, or other drugs that modulate nucleic acid polymerase activity. The method includes providing a primer-template hybrid duplex comprising a nucleic acid template and a nucleic acid primer, wherein either the template or the primer is labeled, or both the template and the primer are labeled; contacting the duplex with a nucleic acid polymerase and a compound; subjecting the hybrid duplex to denaturing conditions; and detecting a signal from the label, wherein a change in the signal compared to a control is indicative that the compound modulates nucleic acid polymerase activity.

### BRIEF DESCRIPTION OF THE DRAWINGS

FIG. 1 depicts a line graph showing the effect of increasing the length of the complementary primer to a 32-mer template (1:1) on the fluorescence ratio after addition of denaturant, where the primer length is increased by addition of the bases shown.

FIG. 2 depicts a line graph showing the effect of increasing the length of the complementary primer to a 78-mer template (1:1) on the fluorescence ratio after addition of denaturant, where the primer length is increased by alternating A and G bases.

FIG. 3 depicts a line graph showing the effect of increasing the length of the complementary primer to a 42-mer template (1:1) on the fluorescence ratio after addition of denaturant, where the primer length is increased by alternating A and G bases.

FIG. 4 depicts a line graph showing the effect of increasing the length of the complementary primer to a 31-mer template (1:1) on the fluorescence ratio after addition of denaturant, where the primer length is increased by alternating A and G bases.

FIG. 5 depicts a line graph showing the results of elongation of 40 nM substrate DNA (15-mer primer) annealed to a 32-mer template (1:2) by Klenow fragment in the presence of 10 mM of each dNTP at 21° C. for 30 min, followed by denaturation with 4 M urea+0.5 M Tris base.

FIG. 6 depicts a line graph showing the effect of reaction time with DNA polymerizing enzyme on the fraction of DNA substrate elongated. The elongation rate was linear until approximately 50% of the substrate was converted to product.

FIG. 7 depicts a line graph showing the relationship between the FRET assay fluorescence intensity ratio and the fraction of DNA substrate elongated to product by DNA polymerizing enzyme, as determined by HPLC size-exclusion chromatography.

### DETAILED DESCRIPTION

The present invention provides methods for assaying the activity of a nucleic acid polymerase. The present invention also provides methods of screening for modulators (inhibitors or activators) of a nucleic acid polymerase.

The method of the invention is based, in part, on the finding that the stability of a double stranded nucleic acid product in the presence of a denaturant can be used as a means for determining the activity of a nucleic acid polymerase. Specifically, the method includes contacting a test nucleic acid polymerase with a primer-template hybrid duplex. The nucleic acid polymerase may, depending on its activity, cause elongation of the primer resulting in the production of a longer double-stranded product. When the product is exposed to a denaturant, hybrid products that have increased length exhibit greater stability than the starting complex (i.e., the starting hybrid duplex molecule) and therefore do not disassociate so rapidly under denaturing conditions. Thus, by labelling the primer-template appropriately, it is possible to determine the activity of the nucleic acid polymerase by detecting for the presence of associated or disassociated primer-template hybrid duplexes in the presence of the denaturant.

Labels

In general, the invention provides a primer oligo(deoxy- or ribo)nucleotide (“primer”) which is annealed to a longer template oligonucleotide to form a hybrid duplex molecule. Each primer and template bears a label, for example, at or near its non-elongating end (i.e., the 5′ end of the primer and the 3′ end of the template).

The primer and/or template can be labelled with any detectable label such as such as ³²P, or any other detectable marker, such as other radioisotopes, a fluorescent compound, an enzyme, or an enzyme co-factor. Examples of radioisotopes include ¹²⁵I, ³⁵S, ¹⁴C, or ³H. Examples of enzymatic labels include horseradish peroxidase, alkaline phosphatase, or luciferase and their detection can be determined by conversion of an appropriate substrate to product.

In one example, the primer and the template can be labelled such that when the labels are in close proximity to each other, a signal is generated. Labeling systems such as these are known in the art and are commercially available. Commercially available systems include fluorescence resonance energy transfer (FRET) or scintillation proximity assay (SPA). By using such a labelling system, the activity of the nucleic acid polymerase can be determined. For example, if the nucleic acid polymerase does not have good activity, once the primer-template hybrid duplex is exposed to the denaturant, the denaturant will cause the duplex to disassociate and no signal will be detectable. Alternatively, if the nucleic acid polymerase is active and causes appropriate elongation of the product, then exposure to the denaturant will not cause the duplex to disassociate and the signal generated from the label will remain detectable.

In another example, the primer and the template can be labelled with a signal quencher such that when the duplex remains associated no signal is generated. However, dissociation of the duplex upon exposure to the denaturant will result in a signal being detectable. Means of labelling and detecting the signal are discussed more fully below.

FRET

If the labelling system is based on the FRET system, either the primer can bear a fluorescent donor label and the template can bear a FRET acceptor label or fluorescence quencher, or the primer can bear a FRET acceptor or fluorescence quencher and the template can bear a fluorescence donor label. FRET acceptor labels may or may not be fluorescent. Elongation of the primer at the 3′ end by polymerase action results in a longer double-stranded molecule. Because of the increased length of the hybrid product, the hybrid product is more stable to denaturation than the starting complex (the starting hybrid duplex molecule), and therefore, more of the fluorescence donor and acceptor/quencher remain in close proximity under denaturing conditions for the generation of a fluorescence signal. If the fluorescence signal consists of reduced donor fluorescence and, if the FRET acceptor is fluorescent, increased acceptor fluorescence may result. Detection of a change in the fluorescence signal after both the enzymatic reaction has occurred and denaturing conditions have been applied, is used to detect polymerase activity.

The FRET signal is typically measured by the ratio of acceptor fluorescence to donor fluorescence for maximal signal:noise ratio. In this case, elongation of the primer by the polymerase results in an increase of the acceptor:donor fluorescence or decrease in the donor:acceptor fluorescence ratio as compared with a control that has not been elongated, after application of denaturing conditions (which includes the addition of a denaturant or chaotropic agent and/or the application of heat). Chaotropic agents that can be used include urea, salts of chaotropic anions such as isothiocyanate, iodide, perchlorate, nitrate, and bromide; salts of chaotropic cations such as guanidinium, barium and calcium; water-miscible organic solvents such as formamide, ethanol, and methanol; bases such as sodium hydroxide, potassium hydroxide and Tris Base; acids; ionic detergents such as sodium dodecyl sulphate. Alternatively, the donor or acceptor fluorescence alone can be measured. If the donor fluorescence alone is measured, elongation of the primer by the polymerase results in a decrease of the fluorescence intensity as compared with a control that has not been elongated, after application of denaturing conditions. If the acceptor fluorescence alone is measured, elongation of the primer by the polymerase results in an increase of the fluorescence intensity as compared with a control that has not been elongated, after application of denaturing conditions. The donor fluorescence may also be measured in the presence of either a non-fluorescent FRET acceptor or fluorescence quencher.

In some embodiments, the fluorescence measurement is the ratio between the FRET acceptor fluorescence and the fluorescence donor fluorescence, or the ratio between the fluorescence donor fluorescence and the FRET acceptor fluorescence.

Protocols for FRET technology, FRET signal detection, and fluorescent/quencher labeling of nucleic acids are well known to the art, and are described in the following: Selvin, 1995, Methods Enzymol., 246:300-334; Lakowicz, Joseph, R, Principles of Fluorescence Spectroscopy, Plenum Press, New York, 1983, Chp. 10 (“Energy Transfer”), pp. 305-341; Waggoner, 1995, Methods Enzymol., 246:362-373.

SPA

Labelling systems based on scintillation proximity assays can also be used. Typically, the primer or the template is immoblized close to a scintillant molecule prior to the addition of the polymerase and the denaturant. Using the SPA approach, the non-immobilizing portion of the primer-template duplex, is labelled with a radioisotope such as a weak β emitter. When the primer-template duplex is associated, the radiolabel is in close proximity to the scintillant such that the energy is absorbed by the scintillant, which in turn emits a signal. However, should the primer-template duplex disassociate, the non-immobilized portion of the primer-template duplex, i.e., the radiolabeled primer or template, will not be located close to the scintillant and the energy from the radiolabel will not cause the scintillant to emit energy.

Using SPA, it is also possible to carry out the invention by first exposing the primer-template duplex to a test nucleic acid polymerase and then following denaturation, the primer or template is captured and immobilized. If the primer-template duplex is still bound to each other following exposure to the denaturant, the radiolabel will cause the scintillant to emit energy and the activity of the test nucleic acid polymerase can be detected.

In one example, the template is immobilized by methods known in the art to a microscopic bead (about 2 500 fit easily on a pinhead) containing a scintillant. The scintillant can be stimulated to emit light upon annealing of a radiolabelled primer to the duplex. This stimulation event only occurs when the radiolabelled primer of interest is bound to the template which in turn is bound to the surface of the bead. In this example, when the primer-template duplex is exposed to a denaturant, depending on the stability of the duplex, a signal may be detected.

Examples of radioisotopes that can be used in the invention include ³H, ³³P, ³⁵S or ¹²⁵I. SPA beads are available commercially. For example, SPA beads can either be made from yttrium silicate (YSi) which has scintillant properties by virtue of cerium ions within the crystal lattice, or polyvinyltoluene (PVT) which acts as a solid solvent for anthrancine.

Protocols for SPA technology, SPA signal detection, and labeling of nucleic acids are well known to the art, and are described in manuals available from Amersham Biosciences UK Limited (Amersham Place, Little Chalfont, Buckinghamshire HP7 9NA, England), the contents of which are incorporated herein by reference. As used herein, conditions permitting elongation of the primer refers to a reaction environment supportive of nucleic acid polymerase activity and supportive of primer extension or elongation. Such conditions will vary for particular nucleic acid polymerases, and are well known to those of skill in the art.

Conditions in which elongation does not occur are any conditions that inhibit primer extension or elongation. Such conditions are typically used in the assays of the present invention for control samples or control reactions. Such conditions will be apparent to those of skill in the art and can include, by way of non-limiting example, any one of the following conditions: the enzyme (the nucleic acid polymerase) is omitted; an inactivated or inactive mutant enzyme is used; the nucleotide substrate is omitted; magnesium ion is omitted, or is chelated by a chelating agent, such as EDTA; an inhibitor of the enzyme is present; a denaturant is added prior to initiation of the reaction, such that the enzyme is inactivated or the primer/template hybrid is melted; the ionic strength or pH conditions are unsuitable for the particular enzyme; DNase is added to destroy the DNA substrate so that the two labels are irrevocably separated.

Methods for detecting a labelled signal are known in the art and are dependent on the type of label being used. By detecting a signal from the label we mean that the signal detected can either be detected directly or indirectly. For example, where the primer or template is labelled with a fluorescent label as when the FRET system is being used, the fluorescent signal can be detected directly. Alternatively, the signal from the label can be detected indirectly, when for example the primer or the template is labelled with a radioisotope using the SPA method, the signal being detected is the energy being emitted by the scintillant.

The methods of the present invention are useful for studying the function of DNA and RNA polymerases. The methods of the present invention can be used for assaying the activity of any enzyme that polymerizes RNA or DNA nucleotides to a primer in a template-directed fashion. Nucleic acid template-dependent polymerases from any source can be assayed using the methods of the present invention, including, but not limited to, viral, bacterial, prokaryotic, eukaryotic, and cancer or disease-associated nucleic acid polymerases. In some embodiments, the nucleic acid polymerase is a DNA polymerase. In some embodiments, the nucleic acid polymerase is a bacterial DNA polymerase. In some embodiments, the nucleic acid polymerase is selected from Escherichia coli DnaE and Haemophilus influenzae DnaE. The methods of the present invention are also useful for testing for modulators (inhibitors or activators) of polymerases, in various formats, including, but not limited to, high-throughput screening of compound libraries and rational drug design.

The design of the primer and template should be carefully considered to maximize the dynamic range, sensitivity and linearity of the assay.

The elongatable region of the template (i.e., the part not initially base-paired to the primer) should be designed so as to avoid self-complementarity. This will avoid the formation of hairpins and/or double-stranded template that could interfere with the ability of the polymerase to elongate the primer.

The primer-binding sequence of the template should also be carefully considered to assure that the primer binds in a well-defined location at the 3′ end of the template. In addition, the sequence and length of the primer should be adjusted so the primer-template affinity is high enough that the two are base-paired under the non-denaturing conditions of the enzymatic reaction, but dissociate completely under the subsequent denaturing conditions. Moreover, the sequence should also be adjusted so that the addition of even a single nucleotide results in an increase in resistance to denaturation. The length of the template should be adjusted so that, for that particular sequence, addition of nucleotides to the primer increases the stability of the duplex over the entire length. In other words, the template should not be so long that addition of nucleotides could not be detected because no further stabilization to denaturation was achieved.

Selection and Location of Labels for DNA

Assays of the present invention utilize primer and template pairs, each of which bears a label. It does not matter whether the donor label is attached to the template or the primer oligonucleotide, as long as the complementary oligonucleotide is labeled with the FRET acceptor/fluorescent quencher.

Any method appropriate for attaching or affixing a label can be used to generate primers and templates that bear labels. For example, the labels can be in the form of labeled nucleotide bases, or a lable can be affixed or attached to any portion of the nucleotide in a primer or template. In some embodiments, the label is in the form of a labeled nucleotide base positioned near the 5′end of the primer and the 3′ end of the template.

Proximity of the donor and acceptor are important for FRET or quenching to occur. The 5′ end of the primer and the 3′ end of the template can be labeled in order to achieve this proximity, and any single-stranded overhang on the labeled end of the substrate should not be too long. We have found that 0-3 nucleotide (nt) overhangs are acceptable, but longer ones are also acceptable.

The choice of fluorescence donor and FRET acceptor/quencher probes (also known as dyes or labels) should be made so that a strong donor fluorescence change occurs when the primer and template are annealed. The probes should also be chemically stable to the denaturing conditions.

Many acceptable label pairs that can be used in the assays of the present invention are known to the art, including a wide variety that are commercially available. Examples of commercially available dyes/labels/probes, including fluorescent dyes and quenchers, useful for labeling oligonucleotides can be found in, for example, the 2002 catalog of TriLink Biotechnologies (San Diego, Calif.).

An example of a useful donor-acceptor pair is fluorescein-rhodamine, although many useful pairs are possible. In some embodiments, the donor-acceptor pair is 5- or 6-carboxyfluorescein (FAM)—5- or 6-carboxy tetramethylrhodamine (TAMRA).

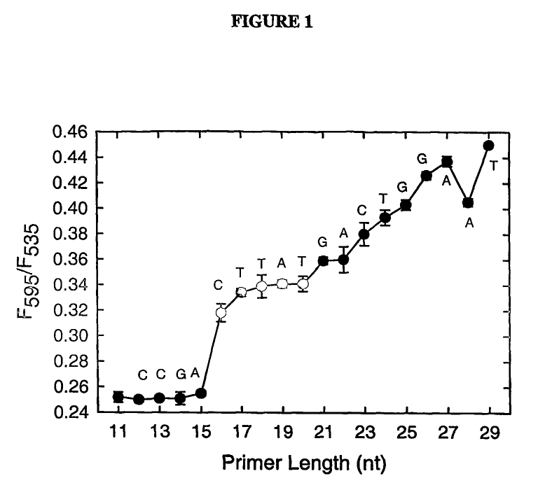

The number of single-stranded spacing nucleotides between the donor and FRET acceptor/fluorescence quencher should be optimized from the point of view of avoiding interference of the probes with annealing of the DNA, but without reducing the strength of the FRET or quenching interaction. For the substrate used in FIG. 1, the donor probe was the fluorescein derivativeFAM and the acceptor probe was the rhodamine deriviativeTAMRA. The spacing between them was varied between 0 and 3 nucleotides, without any significant effect on the strength of the FRET interaction.

The donor label must be fluorescent. The acceptor label does not have to be fluorescent, but its absorbance spectrum must overlap with the fluorescence emission of the donor for FRET to occur. However, if the ratio method is to be used (an example of which is described in Example 8, the acceptor label should be fluorescent.

The Influence of Solution Conditions

The ability of two short, complementary oligonucleotides, such as the template and primer used in the assays of the present invention, to anneal depends on the nucleotide sequence, temperature, ionic strength, and cation concentration. For a given pair of oligonucleotides, annealing decreases with increasing temperature, but increases with increasing ionic strength, and cation concentrations.

Selective denaturation can be achieved by a variety of methods known to the art. Any chaotropic agent can be used to effect selective denaturation of less polymerized substrates, including, but not limited to, chemical denaturants such as urea, guanidine hydrochloride, acid, base, organic solvents such as formamide, or mixtures thereof. Heat may also be used to effect denaturation.

Screening Assays

The methods of the present invention are useful for identifying compounds such as peptides, peptidomimetics, small molecules, or other drugs which modulate (increase or decrease) nucleic acid polymerase activity. Typically, the method includes providing a primer-template hybrid duplex comprising a nucleic acid template and a nucleic acid primer, wherein either the template and/or the primer is labeled. The duplex is then contacted with a nucleic acid polymerase and a compound under conditions that allow the polymerase to elongate the primer. The duplex is then exposed to denaturing conditions and a signal from the label is detected. A change in the signal compared to a control (i.e., where no compound is added) is indicative that the compound modulates nucleic acid polymerase activity. Compounds identified by the methods of the invention can be used in the treatment of diseases including bacterial infections, viral infections, parasitic infections, cancer, and autoimmune diseases.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Method for Optimization of Primer Length

TABLE 1

SEQ
ID
NO

| | | | |
|---|---|---|---|
| | * | | |
| Test template: | 3′-TAMRA-TGTATTGATGACTGGGCTGAA-5′ | | 1 |
| Nested primers: | 5′-FAM-TATCTACTGAC-3′ | 11 nt | 2 |
| | 5′-FAM-TATCTACTGACC-3′ | 12 nt | 3 |
| | 5′-FAM-TATCTACTGACCC-3′ | 13 nt | 4 |
| | 5′-FAM-TATCTACTGACCCG-3′ | 14 nt | 5 |
| | 5′-FAM-TATCTACTGACCCGA-3′ | 15 nt | 6 |
| | 5′-FAM-TATCTACTGACCCGAC-3′ | 16 nt | 7 |
| | 5′-FAM-TATCTACTGACCCGACT-3′ | 17 nt | 8 |
| | 5′-FAM-TATCTACTGACCCGACTT-3′ | 18 nt | 9 |

* = mismatch

TABLE 2
Non-denaturing conditions

| Template: | absent | present | |
|---|---|---|---|
| Primer length | F595/F535 | F595/F535 | base added |
| 11 | 0.20 | 0.28 | — |
| 12 | 0.20 | 0.30 | C |
| 13 | 0.20 | 0.40 | C |
| 14 | 0.20 | 0.49 | G |
| 15 | 0.20 | 0.50 | A |
| 16 | 0.20 | 0.54 | C |
| 17 | 0.20 | 0.52 | T |
| 18 | 0.20 | 0.52 | T |

Conclusion: To achieve full annealing of primer and template under non-denaturing conditions at room temperature, i.e., conditions for enzymatic reaction, the primer should be at least 14 nt long. Note that this length is specific for this design of primer and template sequence, for this temperature, and under these buffer conditions.

TABLE 3
Denaturing conditions (4 M urea, 0.5 M Tris base)

| Template: | Absent | present | |
|---|---|---|---|
| Primer length | F595/F535 | F595/F535 | base added |
| 11 | 0.18 | 0.26 | — |
| 12 | 0.18 | 0.26 | C |
| 13 | 0.18 | 0.26 | C |
| 14 | 0.18 | 0.26 | G |
| 15 | 0.18 | 0.26 | A |
| 16 | 0.18 | 0.30 | C |
| 17 | 0.18 | 0.32 | T |
| 18 | 0.18 | 0.33 | T |

Conclusion: To maximize dissociation of unelongated substrate under denaturing conditions, the primer should not be longer than 15 nt. Note that this length is specific for this design of primer and template sequence, for this temperature, and under these buffer conditions.

Combining the results from non-denaturing and denaturing conditions, the primer should be 14 or 15 nt long.

### Example 2

### Method for Optimization of Primer Length

TABLE 4

SEQ
ID
NO

| | | | |
|---|---|---|---|
| Test template: | 3′-TAMRA-CAACACAACACAGC(TC)₁₄T-5′ | | 10 |
| Nested primers: | 5′-FAM-GTTGTGTTGTGTCG | 14 nt | 11 |
| | 5′-FAM-TTGTGTTGTGTCG | 13 nt | 12 |
| | 5′-FAM-TGTGTTGTGTCG | 12 nt | 13 |
| | 5′-FAM-GTGTTGTGTGG | 11 nt | 14 |
| | 5′-FAM-TGTTGTGTCG | 10 nt | 15 |

TABLE 5

| | |
|---|---|
| Primer | F595/F535 |

| Length | Non-denaturing | Denaturing |
|---|---|---|
| 14 nt | 0.68 | 0.34 |
| 13 nt | 0.65 | 0.31 |
| 12 nt | 0.60 | 0.29 |
| 11 nt | 0.63 | 0.29 |
| 10 nt | 0.46 | 0.28 |

Conclusion: For this primer and template sequence, and under these measurement conditions, the primer should be at least 11 nt long to remain annealed under non-denaturing conditions, and no longer than 12 nt to minimize annealing under denaturing conditions.

### Example 3

### The Effect of Nucleotide Spacer Length Between FAM and TAMRA End Labels on Energy Transfer

Primer:

(SEQ ID NO: 16)

5′-FAM-TATCTACTGACCCGACTTATGACTGGAAT-3′

TABLE 6
Templates with different spacer lengths

| | |
|---|---|
| | SEQ |
| F595/ | ID |
| U535 | NO |

| | | |
|---|---|---|
| 0 nt: 3′-TAMRA-ATTGATGACTGGGCTGAATACTGACCTTA-5′ | 0.44 | 17 |
| 1 nt: 3′-TAMRA-TATTGATGACTGGGCTGAATACTGACCTTA-5′ | 0.43 | 18 |
| 2 nt: 3′-TAMRA-GTATTGATGACTGGGCTGAATACTGACCTTA-5′ | 0.42 | 19 |
| 3 nt: 3′-TAMRA-TGTATTGATGACTGGGCTGAATACTGACCTTA-5′ | 0.45 | 20 |

Conclusion: There was little effect of nucleotide spacer lengths of 0 to 3 nt on energy transfer between the two labels.

### Example 4

### The Effect of Elongation Region Sequence on Increase in FRET Signal

Template:

(SEQ ID NO: 21)

3′-TAMRA-TGTATTGATGACTGGGCTGAATACTGACCTTA-5′

Primer Set Builds From:

(SEQ ID NO: 22)

5′-FAM-TATCTACTGAC

The FRET signal results of carrying out the assay with increasingly longer primers are shown in FIG. 1.

Conclusion: A:T base pairs, with 2 hydrogen bonds, result in smaller increases in stability of the duplex DNA than C:G base pairs, with 3 hydrogen bonds. This is reflected in relatively little increase in F595/F535 for additions of A or T to the primer, and much larger increases for the addition of C or G.

A smoother increase in FRET signal can be achieved by using a template in which the elongation region contains alternating 2-hydrogen bond and 3-hydrogen bond bases, as follows.

Primer:

(SEQ ID NO: 23)

5′-FAM-GTTGTGTTGTGTCG(AG)ₙ-3′
Template:

(SEQ ID NO: 24)

3′-TAMRA-CAACACAACACAG(CT)₃₂C-5′

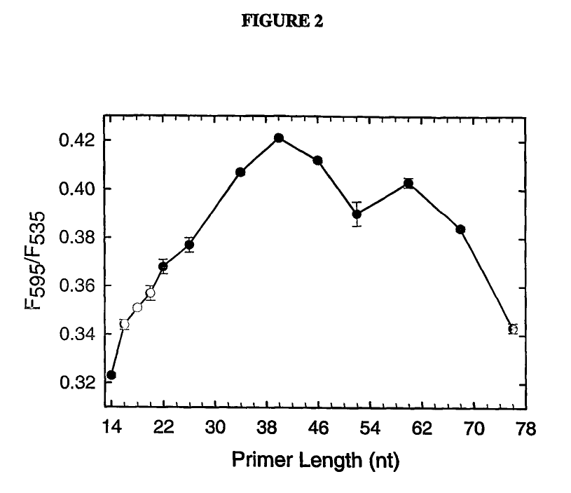

The results are shown in FIG. 2. Note, however, that the FRET signal decreased for longer primer lengths. This may be attributable to slippage of the primer along the template because of the repetitive sequence, which allows multiple possible sites for annealing of longer primers. This undesirable effect can be eliminated by reducing the length of the repetitive elongation region of the template, as in the following Example 5.

### Example 5

### Effect of Template Elongation Region Repetitive Sequence Length on FRET Signal

Template:

(SEQ ID NO: 25)

3-TAMRA-CAACAGAACACAG(CT)₁₅-5′
Primers:

(SEQ ID NO: 26)

5′-FAM-GTGTTGCG-3′

(SEQ ID NO: 27)

5′-FAM-GTGTTGCGA-3′

(SEQ ID NO: 28)

5′-FAM-GTGTTGCG(AG)ₙ-3′

(n = 1-14)

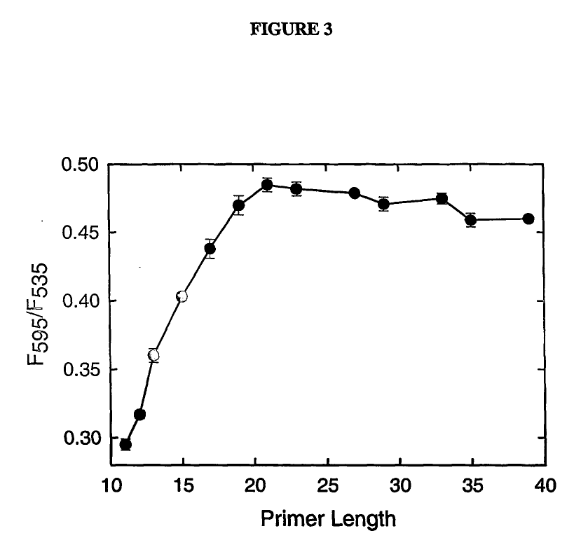

The results are shown in FIG. 3. Notice that in this example, by using a shorter repetitive elongation sequence in the template, the tendency for the FRET signal to decrease as the primer length increases was greatly reduced, as compared with previous Example 4. Moreover, the size of the FRET signal change was larger than in Example 4, which is beneficial for a quantitative assay.

### Example 6

### Minimizing Length of Template Single-Stranded Region

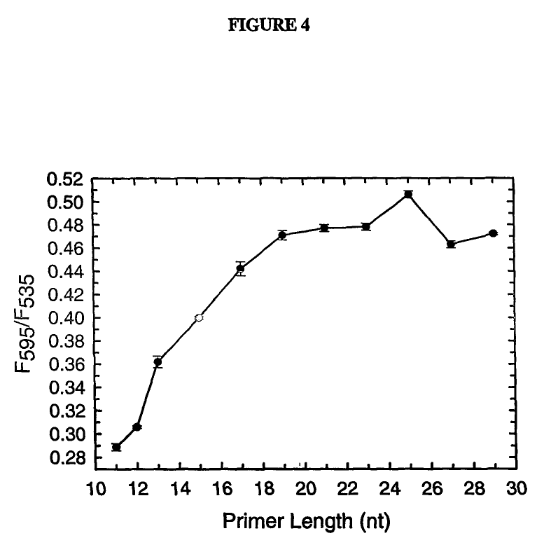

In Example 5, the FRET signal was seen to be maximal after addition of only 10 nucleotides to the primer. This result suggests that the repetitive elongation of the region need not be very long. Using a shorter template (below), the results are shown in FIG. 4.

Template:

(SEQ ID NO: 29)

3-TAMRA-CAACACAACACAG(CT)₈-5′
Primers:

(SEQ ID NO: 30)

5′-FAM-GTGTTGCG-3′

(SEQ ID NO: 31)

5′-FAM-GTGTTGCGA-3′

(SEQ ID NO: 32)

5′-FAM-GTGTTGCG(AG)ₙ-3′

(n = 1-9)

As in Example 5, the maximal FRET signal was obtained by the addition of 10 nt to the primer, annealed to a shorter template. Because of the difficulty and expense of synthesizing oligonucleotides labeled with fluorescent dyes, minimizing the length of the required oligonucleotides provides the advantages of economy and convenience.

### Example 7

### Enzymatic Elongation of the Primer by Escherichia Coli DNA Polymerase I Klenow Fragment

Escherichia coli DNA polymerase I Klenow fragment is a commercially available DNA polymerase that lacks exonuclease activity. It was used to elongate the following DNA substrate in the presence of dATP, dGTP, dCTP, and dTTP, followed by denaturation in 4 M urea, 0.5 M Tris base.

Template:

(SEQ ID NO: 33)

3′-TAMRA-TGTATTGATGACTGGGCTGAATACTGACCTTA-5′
Primer:

(SEQ ID NO: 34)

5′-FAM-TATCTACTGACCCGA-3′

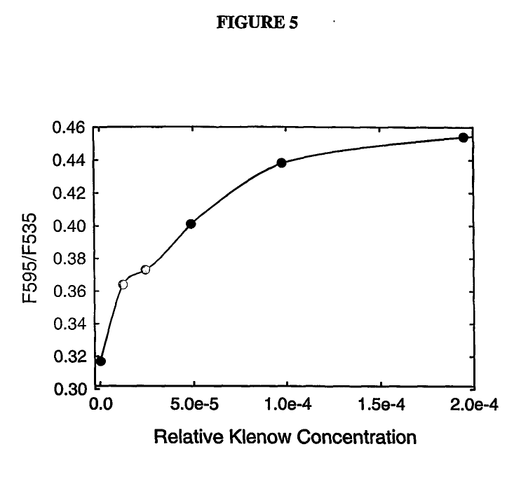

The results are shown in FIG. 5. Conclusion: Enzymatic addition of deoxyribonucleotides to the synthetic FRET assay DNA substrate resulted in an increased FRET signal.

### Example 8

### Enzymatic Elongation of the Primer by Haemophilus influenzae DNA Polymerase III Alpha Subunit (DnaE)

DNA polymerase III is responsible for chromosomal DNA replication in bacteria. The alpha subunit (DnaE, the product of the dnae gene) contains the catalytic active site responsible for addition of deoxyribonucleotides to the 3′ end of the nascent DNA strand. The DNA substrate used in this example is shown below.

Template:

(SEQ ID NO:35)

3-TAMRA-CAACACAACACAGC(TC)₈T-5′
Primer:

(SEQ ID NO:36)

5′-FAM-GTGTTGTGTCG

The reaction contained 32 pM DnaE protein, 140 nM DNA substrate, 40 □M dATP, 40 □M dGTP, and 8 MM MgCl₂ in a buffer composed of 50 mM MOPS-NaOH (pH 7.5), 20% (v/v) glycerol, 10 mM dithiothreitol, 1 mM EDTA, 0.002% (w/v) Brij-35, 4 mM n-octylglucoside, and 100 mM bovine serum albumin. The reaction occurred at 20° C. It was terminated by the addition of an equal volume of denaturing solution composed of 8 M urea, 1 M Tris base, and 50 mM EDTA. The change in the F595/F535 fluorescence ratio was converted to fraction of substrate elongated, as described in Example 9, using the linearization formula:
Fraction Elongated=(□F595/F353)/(0.282+0.915*(□F595/F353))

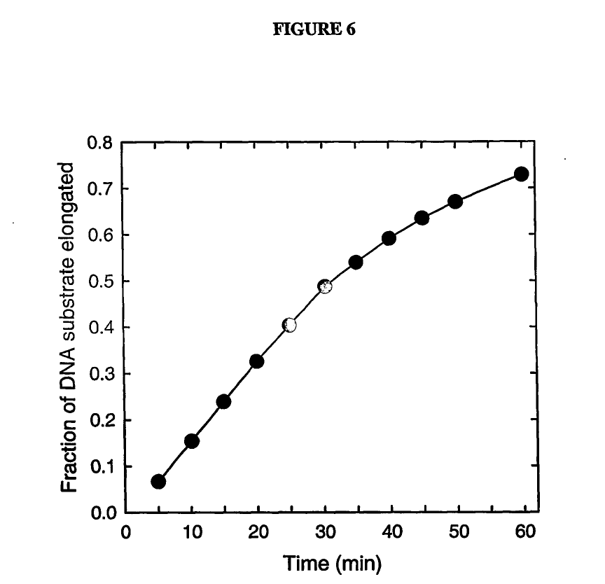

The results are shown in FIG. 6.

### Example 9

### Linearization of FRET Signal F595/F535

In the FRET assay, the template and primer of the substrate DNA, carrying their respective labels, are separated by the denaturant, causing a loss of FRET, whereas the template and primer of the elongated primer remain together, allowing FRET to occur. FRET results in a decrease in donor label (FAM) fluorescence and an increase in acceptor label (TAMRA) fluorescence. After addition of denaturant to the samples, both fluorescent labels were excited by 485 nm light. FAM fluorescence was measured at 535 nm and TAMRA fluorescence was measured at 595 nm. It should be noted that the 595 nm fluorescence is not due entirely to TAMRA, since FAM fluorescence can also be detected at this wavelength. The change in the fluorescence ratio F595/F535 was used to measure the extent of elongation of the DNA substrate, rather than measuring the decrease in F535 or increase in F595 because the ratio is less prone to geometric artifacts (i.e., the height of the liquid in the well of a multiwell plate or the depth of the meniscus), resulting in more precise measurements. Because of the combination of FAM and TAMRA fluorescence intensities at 595 nm, however, the relationship between the change in F595/F535 and the fraction of the substrate that has been converted to product was not linear. The relationship can be shown to follow the hyperbolic relationship: Fraction Elongated=(□F595/F353)/(a+b*(□F595/F353)).

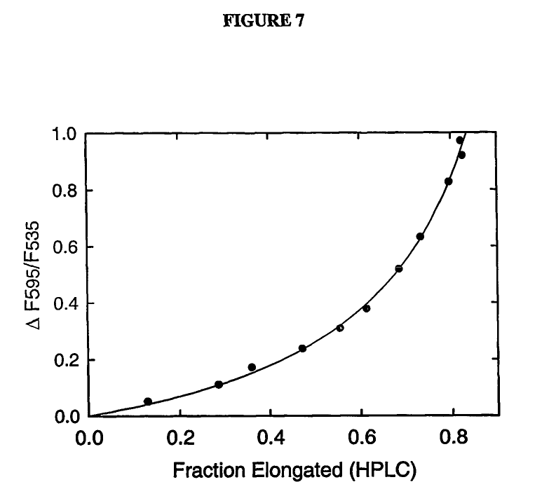

In order to convert from the measured F595/F535 to the desired Fraction Elongated, it is necessary to know the values of a and b. These values were obtained by making an independent measurement of the fraction of substrate converted to product. We used size exclusion chromatography under denaturing conditions to separate the annealed product from the denatured template and primer in samples prepared by reacting the substrate with various amounts of polymerase. The quantity of the FAM-labeled primer was measured by detecting the FAM fluorescence. The reduction of FAM-labeled primer with increasing polymerization was plotted against the measured F595/F535 of the same samples, and the values of a and b determined by fitting the data to the above formula. The results of such an approach, using the substrate from Example 8, are shown in FIG. 7.

### Example 10

### High Throughput Screening for Inhibitors of Haemophilus influenzae DnaE

The FRET assay was used for high throughput screening for inhibitors of H. influenzae DnaE. Into each well of a 384-well black assay plate 3 □l of test compound solution at 100 □M and 20 □l of a 1.5× solution of DNA, dATP, dGTP, and DnaE in buffer were added. The polymerase reaction was initiated by the addition of 7 □l of 34.3 mM MgCl₂ in buffer. After 30 min, the reactions were terminated by the addition of 30 □l of denaturant. After a minimum of 15 minutes, the signal in each well was measured. The signal consisted of the ratio of fluorescence at 595 nm and 535 nm when an excitation wavelength of 485 nm was used. Measurements were made with a Tecan Ultra plate reader. The excitation filter was a 485 nm fluorescence filter with a 20 nm bandpass. The emission filters were a 535 nm fluorescence filter with a 25 nm bandpass and a 595 nm fluorescence filter with a 35 nm bandpass. For each compound, a value of % inhibition was calculated. As a 100% inhibition control, 100 mM sodium pyrophosphate was used. Using this method inhibitors of H. influenzae DnaE were identified.

The foregoing examples are meant to illustrate the invention and are not to be construed to limit the invention in any way. Those skilled in the art will recognize modifications that are within the spirit and scope of the invention.

We claim:

1. A method of detecting nucleic acid polymerase activity, the method comprising:
a) providing a primer-template hybrid duplex comprising a nucleic acid template and a nucleic acid primer, wherein the template, the primer, or both the template and the primer comprise a label;
b) contacting the duplex with a nucleic acid polymerase;
c) subjecting the hybrid duplex to denaturing conditions; and
d) detecting a signal from the label, wherein a change in the signal compared to a control is indicative of nucleic acid polymerase activity.

2. The method of claim 1, wherein the nucleic acid template comprises a first label and the template comprises a second label.

3. The method of claim 2, wherein the first label is a fluorescence donor and the second label is a FRET acceptor or fluorescence quencher.

4. The method of claim 2, wherein the first label is a FRET acceptor or fluorescence quencher and the second label is a fluorescence donor.

5. The method of claim 1, wherein nucleic acid template is immobilized close to a scintillant molecule and the primer is labeled with a radioisotope.

6. The method of claim 1, wherein is primer is immobilized close to a scintillant molecule and the nucleic acid template is labeled with a radioisotope.

7. The method of claim 1 wherein the nucleic acid polymerase is a DNA polymerase.

8. The method of claim 1 wherein the DNA polymerase is a bacterial DnaE.

9. The method of claim 1 wherein the DNA polymerase is E. coli DnaE or H. influenzae DnaE.

10. The method of claims 3 or 4 wherein the fluorescence donor is 5- or 6-carboxyfluorescein (FAM) and the FRET acceptor is 5- or 6-carboxytetramethylrhodamine (TAMRA).

11. The method of claim 1 wherein the denaturing conditions are achieved by application of heat.

12. The method of claim 1 wherein the denaturing conditions are achieved by addition of a chaotropic agent.

13. The method of claim 12 wherein the chaotropic agent is urea.

14. The method of claim 1 wherein the first label is borne at the 5′ end of the primer and the second label is borne at the 3′ end of the template.

15. The method of claim 1 wherein the primer is at least 6 nucleotides in length and the template is at least 10 nucleotides in length.

16. A method of screening for compounds that modulate nucleic acid polymerase activity, the method comprising:
a) providing a primer-template hybrid duplex comprising a nucleic acid template and a nucleic acid primer, wherein either the template or the primer is labeled, or both the template and the primer are labeled;
b) contacting the duplex with a nucleic acid polymerase and a compound;
c) subjecting the hybrid duplex to denaturing conditions; and
e) detecting a signal from the label, wherein a change in the signal compared to a control is indicative that the compound modulates nucleic acid polymerase activity.
